SEQUENCE LISTING (10 sequences)

SEQ ID NO: 1 - DNA, 10353 bp, Homo sapiens
taatcctagcactttgggaggccaggtgggcagatcacttgagtcagaagttcgaaaccagcctggtcaacgtagtgaaaccccatctctactaaaaatacaaaaaatttagccaggcgtggtggcgcacgcctataataccagctactcgggaggctgaggcaggagaattgcttgaacccgggaggcagatgttgcagtgagccgagatcacgccactgcactccagcctgggtgacagagtgatactacaccccccaaaaataaaataaaataaataaatacaactttttgagttgttagcaggtttttcccaaatagggctttgaagaaggtgaatatagaccctgcccgatgccggctggctaggaagaaaggagtgagggaggctgctggtgtgggaggcttgggagggaggcttggcataagtgtgataattggggctggagatttggctgcatggagcagggctggagaactgaaagggctcctatagattattttcccccatatcctgccccaatttgcagttgaagaatcctaagctgacaaaggggaaggcatttactccaggttacactgcagcttagagcccaataacctggtttggtgattccaagttagaatcatggtcttttggcagggtctcgctctgttgcccaggctggagtgcagtgacataatcatggctcactgtatccttgaccttctttctgggctcaagcaatcctcccacctcggcctcccaaagtgctaagattacaggaatgagccaccatacctggccctgaatcttgggtcttggccttagtaattaaaaccaatcaccaccatccgttgcggacttacaacctacagtgttctaaacattttatatgtttgatctcatttaatcctcacatcaatttagggacaaagagccccccaccccccgtttttttttttacagctgaggaaacacttcaaagtggtaagacatttgcccgaggtcctgaaggaagagagtaaagccatgtctgctgttttctagaggctgctactgtcccctttactgccctgaagattcagcctgcggaagacagggggttgccccagtggaattccccagccttgcctagcagagcccattccttccgcccccagatgaagcagggagaggaagctgagtcaaagaaggctgtcagggagggaaaaagaggacagagcctggagtgtggggaggggtttggggaggatatctgacctgggagggggtgttgcaaaaggccaaggatgggccagggggatcattagtttcagaaagaagtctcagggagtcttccatcactttcccttggctgaccactggaggctttcagaccaagggatgggggatccctccagcttcatccccctccctccctttcatacagttcccacaagctctgcagtttgcaaaaccctacccctcccctgagggcctgcggtttcctgcgggtctggggtcttgcctgacttggcagtggagactgcgggcagtggagagaggaggaggtggtgtaagccctttctcatgctggtgctgccacacacacacacacacacacacacacacacacacacacacacacaccctgacccctgagtcagcacttgcctgtcaaggaggggtggggtcacaggagcgcctccttaaagcccccacaacagcagctgcagtcagacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggtgggcaaacacctagtctagagttggggagggctgtccgtgagggtgttgagtgtcccagagaggatgcagggcctcagaggagatgctttaggggtgtgttggtggtgatgggcgtatctgaagaacagaggtgtccagggttaggcagtggggggtcttgtggaggctttgagcagtgatggccagaaatgggcaatggggctttcctaggtgggaaatgggaaatggtttggggtgggggaggcattggagggttctggggtaagcataggctgggagtgaacaggggcaaaccttatgcagctgtggggtagaaatgggctagaggcatccaggggtgagaaggagctgaggatgtctaaggaggggagatccctgggtggtcagaaagcactggtgtctggaaagcatttaatgctttattaaatgttagtccctgctgggcatgacggctcacacttgtaatcccagcactttgggaggctgaggtggtaggatcgctgaagctcaggagtttgagcccagcctaggcaacatagtaagatcctgtctctacaaaaaaattaaagaaatagccaggcacagtgatgtgcacctgtagttccagctatgcagaaggctgagatgggaggatcgcttgagtccaggaggtccaggctgcagtgggctgataccgtctctccgaaaaagaaaaagaaaaaagactccctccatgagtgtctggagggagtcctttggccccagctgggcagagaaaggggtcagagatctggcatgtgtgtgtcccttcatccacaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggtgagccggggccgtgggggcagcggggtggggcggggaggccaggtctggctcttgggccagcggtgaacatgtcctgtcttggacgcgtccctgggtttcactatttaatgtgtggcccctggggagtgtccccacctctgagcctctgtttctccttcagggaaatggctcttgcaatccaagtcctcctgccagggccattgtgagggtctaagtagacaaaaaaaaaaaaaaaaaaaacagtctggaagcaatttatagatgagagcgtggacggcagagagcattgtgtatgttgaagtctctgcgatatggggtgtccctgctgccccgctccagcctttcacttctgacctccttcctctggctcttacgctacaggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcgggtgagaacgtgaggagggaaaatccaagagacctgggcggggtcagggaagggaggaccacggagagcgtggaggcagcagtggccccggcttcctcttgcctgcccgcgctgccctggcttatacggcccctcctgccagacagtgcacagggccagggcgccaggctgggagagcttcgcgcaggcgggatttcagcccgcacttatttcggagcccttgccttgggcagcgcacaatctgcgcagcagtactcggctaaccctcttcctctcgacctgtttcttcagagcacggagacgggtatcccttcgacgggaaggacgggctcctggcacacgcctttcctcctggccccggcattcagggagacgcccatttcgacgatgacgagttgtggtccctgggcaagggcgtcggtgagattctgagtcctcctggcccctgattcccttcattctctcccactcatcacccgccgccctaactccggtcccccctcctcctgcagtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagtgagtgagggggctcgccgagggctgggggcgcccaccacccttgatggtcctgggttctaattccagctctgccactagtgctgtgtggcctgcaattcaccctcccgcactctgggcccaattttctcatctgagaaatgatgagagatgggatgaactgcagaccatccatgggtcaaagaacaggacacacttgggggttataatgtgctgtctccgccttctccccctttcccacatcctcctcgccccaggactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgaggtacctccaccctgtctaccaggttcagccccgccctctcatcatgtattggcccccaaaacgcggctcttccctcccatcagtttgtctttccactctcattggtcctcaggacgaccgtgactccgcccacctacaccacatttccaccactatccctgacttccaatggccccgccccagccactaaggttcggccttttctgcccagctggccgcctcttccttggtctggtgtcccaggcaccgcccacgggtctagcctcttctcaggagtgctctacagcgccccctaggccaccaagattgtttagctccctgtcgggtcggcccctgactccttattggactcatccatctggctcatccaaggccttgggtctctccagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggtaggcgtggtcccgcggctccggggctggggttcccggcagtggtggtggtggggtggccagggctgggggctcggcccggcgctcacgtctcaggctccctctccctccaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtgaggcaggggcagggatgggaggaggaggggaaagggcgtggctgtgccacagtaccaaagaattgggggttggggatcgggggaggaacggggcgtgcaggagaggtgggacctcaacgtctgtctggaagcagagcctgggcccagtcgctgccatgtcagtgcttagaggtggtgataaagagactctagagagagataggtgtgacttcaaaagccagtctactctgggcatggtggctcacgcctctaatcccagggctttgggagacccaaggcgggaggattgcttaagcccaggagttccagaccagcctcggcaacatagccagactcccatctctacaaaaaataaatgagcaaggcgtgaaggcacatgtctgtagtcctagctactctggaggctgaggtgggaggatctcttgagcccaggagttcgaggctgtagtgagctatgattgcaccactgcattccatcctgggccatagaggatgtcgcttaaaacgaaaaagaagaagaagaaagtcctgtggtttgggaagggaggctgagtgaggaggggcctgtgtgccagaggaggcttcactgagaagcttaggggagcagatgttctaggggtacagaggtatgcaggaataggaagagtctcaccccgtgtctctttttaggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggtgaggaggcggggttgtgtggatgcgggagggggctttgcggaggggctgcccgtcccttcccgcccactggccctgtgtccaaggcttagagcccgtcctttccctcctcgctttctcaggaagtactggcgattctctgagggcagggggagccggccgcagggccccttccttatcgccgacaagtggcccgcgctgccccgcaagctggactcggtctttgaggagcggctctccaagaagcttttcttcttctctggttagttacctactttccctcccccgcccggtcaatccccatcagtcaaggaggctcaagagaccatcgataacccacgaaacgtcttgtgcgttttagaaaaatacgccccctggcggacgcagtttagcaaacgtaggggcggctgagtttctgccccctcctctccacgccctcgcgtcgctctacccagcgcctctgcccctgggttgcagggactgcgggcacgcgggctaggaaaggcctcgccggaatctccctcctcgcgttctaggagtacgtgctccctctgcgcccccaaaccgacgtgaccctcctcccctgcagggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggtgagcgccgccgcggccgccggcagggggagcccgggcgccgtcggtccgtccgctagccggctcagcacctgtctcctccgcgcctgcccgcaggttcgacgtgaaggcgcagatggtggatccccggagcgccagcgaggtggaccggatgttccccggggtgcctttggacacgcacgacgtcttccagtaccaaggtgagggctgaggaggatcccttcgtgagacaccacactaagctcctcttagtgagtggtcaaattctgagcgaggaagaaaaagcccttggaaatggaaacaaatgccccagcacagacaagatcccagcagaggcagaggccttctccaggtcatttaggaagtcagggatgcaaccaagaccaggacccagatttcctgcctccccggctggaagctctttctccttcagtacaggacggcaggtggtttgtatggaagctcagttattagacaacagtcatcaagtgccgataatgtgccaggcactgtgctacagggagagataagacaattcacagctctgtgactttgggcaagtcactgcttctctactcttcgagcctcagtttccccatctgtaatatggggactatagctggaattacacttgacttccctttcttaccagtcacatccaaacagttgacaaggtgaacaagatttcctgccaccaaaatctttttcgagtctgtcattttttttgccatcttctttataaacaccccagcccaaaccatactggctgtccaggacctttaacaaattccatgagattagagagggggtaggagtgaagggcaatggtcttgggagtgaccccagatgaattccaaggtcaaagaaattaagaggatctgacactccacccccgtgttctcatctcttcccactcctcctgttatttactctgctccacccacactggctgctctttgaacagatcaaggtcattcctagcttacagcctttgtgccagttgttccctctgtctggaaagcttcccctccagattgtcactgggccatcccactgtcttccttcaggtttcagtgctaaggccattgcttcaatgaggccttctttgatgcttattatctatttacttgtttttattttctccatagctttctatattttctttttttttcttttttctttttttttttttttgagatggagtcttgctctgtcgcccaggctggagtgcagtggcacgatctcagctcactgcaacctccgcctcccgggttcaagcgattctcctgcctcagcctcccaagtagctgggattacaggtgcctgccaccacgcttggctaattttttgtattttttagtagagacggggtttcaccatcttggccaggctggtcttgaactcctgacctcgtgatccacccgcctcagcctcccaaagtgctgggattacaggcatgagccaccgcacccagccgctttctatattttcaaaaccaatctcatttatttatgtgtttgcttaattgtctcttgcctcactagagtgtaagcaccaagataattgagatcatgcctgcattttttctgcttatccccagtatcttgaacaaagcacatagtagatgctcagtaaatgatgaatgaacagatttgttcaatgaatgagcgttgaatgaattgttctgagcattaagatagttggtctattcatttgttaattcattcacaaaatgtgtatggtgtacctactgtgtgctaggctctgtggcagtgctttgggcactgaggtctgtgccctccagcatctcacagaacctcacagcatctcacaggttggggggatggaggtgatatgtgaaaaccttagaaagttctagaaatggcagaagagatggttgtcaagatcttgttcctatttctgtatatgtgggagaattagaatcactcctcttatgcctgcctgtctcctgcagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttagaagcagactttatttatatatgtatgcacgtatgtatgcatgtatgtatttaactgatagagtgcaaaaaaaaaaaaaaaaaggaaaaacaaataactgatagagtgctttctacgtgccagaaagtgttctaggccgggcacggtagctcactcctagcactttgggaggccgaggcaggcggatcacgaggtcaggagattgagaccaccctggctaacacgatgaaaccctgtctctactaaaaaaaaaatagaaaaaattagccgggcgtggtggcgggcgcctgtagtcccagctacttgggaggctgaggcaggagaatggcttgaacctgggaggtggagcttgcagtgagccgagatcacgccactgcactccagcctgggaggtggagcttgcagtgagccgagatcacgccactgcactccagcctgggtgactgagcaagactccgtctcaaaaaaaaaaaaaatagtgttctaggcactttgtaaatgttaacatattcaatcattcctgttaggaaagtatgatgtgattatttctattttacagtcaaggaaatgatcaacctgtttattcattcatcaaacatttattgagcccctacatggagccaggccctgtactgggcaatggggatagagaaatgagttagactttagaatgcataagattccccttggaacttgcttaaaatgcaactccaggctccacctccagagagtctgacctatttacaagggtgattctatggctggtggcggtgggatcacacttggggagtgtgcagtgcatgatcttttattctacaataatggtgtgtgtgtgtgcacatgtgtgtgtgtctgtgttgtggttgaggtccaggaacgtttctcagtcaagatgacatctgaaccggaactgaatcagaaaggatgaacgagcttcttcctgtgcaagggacaatcttcttacagggtaatttaccaagaaccactaaacctaaaaat

SEQ ID NO: 2 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYQEKAYFCQDRFYWRVSSRSELNQVDQVGYVTYDILQCPED

SEQ ID NO: 3 - DNA, 10353 bp, Homo sapiens
taatcctagcactttgggaggccaggtgggcagatcacttgagtcagaagttcgaaaccagcctggtcaacgtagtgaaaccccatctctactaaaaatacaaaaaatttagccaggcgtggtggcgcacgcctataataccagctactcgggaggctgaggcaggagaattgcttgaacccgggaggcagatgttgcagtgagccgagatcacgccactgcactccagcctgggtgacagagtgatactacaccccccaaaaataaaataaaataaataaatacaactttttgagttgttagcaggtttttcccaaatagggctttgaagaaggtgaatatagaccctgcccgatgccggctggctaggaagaaaggagtgagggaggctgctggtgtgggaggcttgggagggaggcttggcataagtgtgataattggggctggagatttggctgcatggagcagggctggagaactgaaagggctcctatagattattttcccccatatcctgccccaatttgcagttgaagaatcctaagctgacaaaggggaaggcatttactccaggttacactgcagcttagagcccaataacctggtttggtgattccaagttagaatcatggtcttttggcagggtctcgctctgttgcccaggctggagtgcagtgacataatcatggctcactgtatccttgaccttctttctgggctcaagcaatcctcccacctcggcctcccaaagtgctaagattacaggaatgagccaccatacctggccctgaatcttgggtcttggccttagtaattaaaaccaatcaccaccatccgttgcggacttacaacctacagtgttctaaacattttatatgtttgatctcatttaatcctcacatcaatttagggacaaagagccccccaccccccgtttttttttttacagctgaggaaacacttcaaagtggtaagacatttgcccgaggtcctgaaggaagagagtaaagccatgtctgctgttttctagaggctgctactgtcccctttactgccctgaagattcagcctgcggaagacagggggttgccccagtggaattccccagccttgcctagcagagcccattccttccgcccccagatgaagcagggagaggaagctgagtcaaagaaggctgtcagggagggaaaaagaggacagagcctggagtgtggggaggggtttggggaggatatctgacctgggagggggtgttgcaaaaggccaaggatgggccagggggatcattagtttcagaaagaagtctcagggagtcttccatcactttcccttggctgaccactggaggctttcagaccaagggatgggggatccctccagcttcatccccctccctccctttcatacagttcccacaagctctgcagtttgcaaaaccctacccctcccctgagggcctgcggtttcctgcgggtctggggtcttgcctgacttggcagtggagactgcgggcagtggagagaggaggaggtggtgtaagccctttctcatgctggtgctgccacacacacacacacacacacacacacacacacacacacacacacaccctgacccctgagtcagcacttgcctgtcaaggaggggtggggtcacaggagcgcctccttaaagcccccacaacagcagctgcagtcagacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggtgggcaaacacctagtctagagttggggagggctgtccgtgagggtgttgagtgtcccagagaggatgcagggcctcagaggagatgctttaggggtgtgttggtggtgatgggcgtatctgaagaacagaggtgtccagggttaggcagtggggggtcttgtggaggctttgagcagtgatggccagaaatgggcaatggggctttcctaggtgggaaatgggaaatggtttggggtgggggaggcattggagggttctggggtaagcataggctgggagtgaacaggggcaaaccttatgcagctgtggggtagaaatgggctagaggcatccaggggtgagaaggagctgaggatgtctaaggaggggagatccctgggtggtcagaaagcactggtgtctggaaagcatttaatgctttattaaatgttagtccctgctgggcatgacggctcacacttgtaatcccagcactttgggaggctgaggtggtaggatcgctgaagctcaggagtttgagcccagcctaggcaacatagtaagatcctgtctctacaaaaaaattaaagaaatagccaggcacagtgatgtgcacctgtagttccagctatgcagaaggctgagatgggaggatcgcttgagtccaggaggtccaggctgcagtgggctgataccgtctctccgaaaaagaaaaagaaaaaagactccctccatgagtgtctggagggagtcctttggccccagctgggcagagaaaggggtcagagatctggcatgtgtgtgtcccttcatccacaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggtgagccggggccgtgggggcagcggggtggggcggggaggccaggtctggctcttgggccagcggtgaacatgtcctgtcttggacgcgtccctgggtttcactatttaatgtgtggcccctggggagtgtccccacctctgagcctctgtttctccttcagggaaatggctcttgcaatccaagtcctcctgccagggccattgtgagggtctaagtagacaaaaaaaaaaaaaaaaaaaacagtctggaagcaatttatagatgagagcgtggacggcagagagcattgtgtatgttgaagtctctgcgatatggggtgtccctgctgccccgctccagcctttcacttctgacctccttcctctggctcttacgctacaggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcgggtgagaacgtgaggagggaaaatccaagagacctgggcggggtcagggaagggaggaccacggagagcgtggaggcagcagtggccccggcttcctcttgcctgcccgcgctgccctggcttatacggcccctcctgccagacagtgcacagggccagggcgccaggctgggagagcttcgcgcaggcgggatttcagcccgcacttatttcggagcccttgccttgggcagcgcacaatctgcgcagcagtactcggctaaccctcttcctctcgacctgtttcttcagagcacggagacgggtatcccttcgacgggaaggacgggctcctggcacacgcctttcctcctggccccggcattcagggagacgcccatttcgacgatgacgagttgtggtccctgggcaagggcgtcggtgagattctgagtcctcctggcccctgattcccttcattctctcccactcatcacccgccgccctaactccggtcccccctcctcctgcagtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagtgagtgagggggctcgccgagggctgggggcgcccaccacccttgatggtcctgggttctaattccagctctgccactagtgctgtgtggcctgcaattcaccctcccgcactctgggcccaattttctcatctgagaaatgatgagagatgggatgaactgcagaccatccatgggtcaaagaacaggacacacttgggggttataatgtgctgtctccgccttctccccctttcccacatcctcctcgccccaggactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgaggtacctccaccctgtctaccaggttcagccccgccctctcatcatgtattggcccccaaaacgcggctcttccctcccatcagtttgtctttccactctcattggtcctcaggacgaccgtgactccgcccacctacaccacatttccaccactatccctgacttccaatggccccgccccagccactaaggttcggccttttctgcccagctggccgcctcttccttggtctggtgtcccaggcaccgcccacgggtctagcctcttctcaggagtgctctacagcgccccctaggccaccaagattgtttagctccctgtcgggtcggcccctgactccttattggactcatccatctggctcatccaaggccttgggtctctccagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggtaggcgtggtcccgcggctccggggctggggttcccggcagtggtggtggtggggtggccagggctgggggctcggcccggcgctcacgtctcaggctccctctccctccaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtgaggcaggggcagggatgggaggaggaggggaaagggcgtggctgtgccacagtaccaaagaattgggggttggggatcgggggaggaacggggcgtgcaggagaggtgggacctcaacgtctgtctggaagcagagcctgggcccagtcgctgccatgtcagtgcttagaggtggtgataaagagactctagagagagataggtgtgacttcaaaagccagtctactctgggcatggtggctcacgcctctaatcccagggctttgggagacccaaggcgggaggattgcttaagcccaggagttccagaccagcctcggcaacatagccagactcccatctctacaaaaaataaatgagcaaggcgtgaaggcacatgtctgtagtcctagctactctggaggctgaggtgggaggatctcttgagcccaggagttcgaggctgtagtgagctatgattgcaccactgcattccatcctgggccatagaggatgtcgcttaaaacgaaaaagaagaagaagaaagtcctgtggtttgggaagggaggctgagtgaggaggggcctgtgtgccagaggaggcttcactgagaagcttaggggagcagatgttctaggggtacagaggtatgcaggaataggaagagtctcaccccgtgtctctttttaggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggtgaggaggcggggttgtgtggatgcgggagggggctttgcggaggggctgcccgtcccttcccgcccactggccctgtgtccaaggcttagagcccgtcctttccctcctcgctttctcaggaagtactggcgattctctgagggcagggggagccggccgcagggccccttccttatcgccgacaagtggcccgcgctgccccgcaagctggactcggtctttgaggagcggctctccaagaagcttttcttcttctctggttagttacctactttccctcccccgcccggtcaatccccatcagtcaaggaggctcaagagaccatcgataacccacgaaacgtcttgtgcgttttagaaaaatacgccccctggcggacgcagtttagcaaacgtaggggcggctgagtttctgccccctcctctccacgccctcgcgtcgctctacccagcgcctctgcccctgggttgcagggactgcgggcacgcgggctaggaaaggcctcgccggaatctccctcctcgcgttctaggagtacgtgctccctctgcgcccccaaaccgacgtgaccctcctcccctgcagggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggtgagcgccgccgcggccgccggcagggggagcccgggcgccgtcggtccgtccgctagccggctcagcacctgtctcctccgcgcctgcccgcaggttcgacgtgaaggcgcagatggtggatccccggagcgccagcgaggtggaccggatgttccccggggtgcctttggacacgcacgacgtcttccagtaccgaggtgagggctgaggaggatcccttcgtgagacaccacactaagctcctcttagtgagtggtcaaattctgagcgaggaagaaaaagcccttggaaatggaaacaaatgccccagcacagacaagatcccagcagaggcagaggccttctccaggtcatttaggaagtcagggatgcaaccaagaccaggacccagatttcctgcctccccggctggaagctctttctccttcagtacaggacggcaggtggtttgtatggaagctcagttattagacaacagtcatcaagtgccgataatgtgccaggcactgtgctacagggagagataagacaattcacagctctgtgactttgggcaagtcactgcttctctactcttcgagcctcagtttccccatctgtaatatggggactatagctggaattacacttgacttccctttcttaccagtcacatccaaacagttgacaaggtgaacaagatttcctgccaccaaaatctttttcgagtctgtcattttttttgccatcttctttataaacaccccagcccaaaccatactggctgtccaggacctttaacaaattccatgagattagagagggggtaggagtgaagggcaatggtcttgggagtgaccccagatgaattccaaggtcaaagaaattaagaggatctgacactccacccccgtgttctcatctcttcccactcctcctgttatttactctgctccacccacactggctgctctttgaacagatcaaggtcattcctagcttacagcctttgtgccagttgttccctctgtctggaaagcttcccctccagattgtcactgggccatcccactgtcttccttcaggtttcagtgctaaggccattgcttcaatgaggccttctttgatgcttattatctatttacttgtttttattttctccatagctttctatattttctttttttttcttttttctttttttttttttttgagatggagtcttgctctgtcgcccaggctggagtgcagtggcacgatctcagctcactgcaacctccgcctcccgggttcaagcgattctcctgcctcagcctcccaagtagctgggattacaggtgcctgccaccacgcttggctaattttttgtattttttagtagagacggggtttcaccatcttggccaggctggtcttgaactcctgacctcgtgatccacccgcctcagcctcccaaagtgctgggattacaggcatgagccaccgcacccagccgctttctatattttcaaaaccaatctcatttatttatgtgtttgcttaattgtctcttgcctcactagagtgtaagcaccaagataattgagatcatgcctgcattttttctgcttatccccagtatcttgaacaaagcacatagtagatgctcagtaaatgatgaatgaacagatttgttcaatgaatgagcgttgaatgaattgttctgagcattaagatagttggtctattcatttgttaattcattcacaaaatgtgtatggtgtacctactgtgtgctaggctctgtggcagtgctttgggcactgaggtctgtgccctccagcatctcacagaacctcacagcatctcacaggttggggggatggaggtgatatgtgaaaaccttagaaagttctagaaatggcagaagagatggttgtcaagatcttgttcctatttctgtatatgtgggagaattagaatcactcctcttatgcctgcctgtctcctgcagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttagaagcagactttatttatatatgtatgcacgtatgtatgcatgtatgtatttaactgatagagtgcaaaaaaaaaaaaaaaaaggaaaaacaaataactgatagagtgctttctacgtgccagaaagtgttctaggccgggcacggtagctcactcctagcactttgggaggccgaggcaggcggatcacgaggtcaggagattgagaccaccctggctaacacgatgaaaccctgtctctactaaaaaaaaaatagaaaaaattagccgggcgtggtggcgggcgcctgtagtcccagctacttgggaggctgaggcaggagaatggcttgaacctgggaggtggagcttgcagtgagccgagatcacgccactgcactccagcctgggaggtggagcttgcagtgagccgagatcacgccactgcactccagcctgggtgactgagcaagactccgtctcaaaaaaaaaaaaaatagtgttctaggcactttgtaaatgttaacatattcaatcattcctgttaggaaagtatgatgtgattatttctattttacagtcaaggaaatgatcaacctgtttattcattcatcaaacatttattgagcccctacatggagccaggccctgtactgggcaatggggatagagaaatgagttagactttagaatgcataagattccccttggaacttgcttaaaatgcaactccaggctccacctccagagagtctgacctatttacaagggtgattctatggctggtggcggtgggatcacacttggggagtgtgcagtgcatgatcttttattctacaataatggtgtgtgtgtgtgcacatgtgtgtgtgtctgtgttgtggttgaggtccaggaacgtttctcagtcaagatgacatctgaaccggaactgaatcagaaaggatgaacgagcttcttcctgtgcaagggacaatcttcttacagggtaatttaccaagaaccactaaacctaaaaat

SEQ ID NO: 4 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 5 - DNA, 552 bp, Homo sapiens
ttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccaagagaaagcctatttctgccaggaccgcttctactggcgcgtgagttcccggagtgagttgaaccaggtggaccaagtgggctacgtgacctatgacatcctgcagtgc

SEQ ID NO: 6 - protein, 184 aa, Homo sapiens
FDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYQEKAYFCQDRFYWRVSSRSELNQVDQVGYVTYDILQC

SEQ ID NO: 7 - DNA, 552 bp, Homo sapiens
ttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgc

SEQ ID NO: 8 - protein, 184 aa, Homo sapiens
FDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQC

SEQ ID NO: 9 - DNA, 51 bp, artificial
  Probe  Sequence  A at position 26
gacacgcacgacgtcttccagtaccaaggtgagggctgaggaggatccctt

SEQ ID NO: 10 - DNA, 51 bp, artificial
  Probe Sequence  G at position 26
gacacgcacgacgtcttccagtaccgaggtgagggctgaggaggatccctt

(19) United States
(12) Patent Application Publication
(10) Pub. No.: US 2011/0035818 A1
(43) Pub. Date: Feb. 10, 2011

(54) DIAGNOSTIC MARKER AND PLATFORM FOR DRUG DESIGN IN MYOCARDIAL INFARCTION AND HEART FAILURE

(75) Inventor: Daniel R. WAGNER, Bertrange (LU)

Correspondence Address: QUINE INTELLECTUAL PROPERTY LAW GROUP, P.C., P O BOX 458, ALAMEDA, CA, 94501, US

(73) Assignee: Centre de Recherche Public de la Santé

(21) Appl. No.: 12/448,929

(22) PCT Filed: Jan. 15, 2008
(86) PCT No.: PCT/EP2008/000657

§ 371 Date: Dec. 8, 2009

Related U.S. Application Data
(60) Provisional application No. 60/884,979, filed on Jan. 15, 2007.

(51) Int. Cl. A01K 67/027; C12Q 1/37; C12Q 1/68; G01N 33/573; G01N 33/50; C12N 15/63; C12N 5/071; A61P 9/04

(52) U.S. Cl. 800/9; 435/23; 435/6; 435/7.4; 436/94; 435/320.1; 435/325

(57) ABSTRACT

Methods for determining the susceptibility of an individual to a heart condition, post myocardial infarction, comprising detecting the presence of an amino acid change in the sequence of the hemopexin domain of MMP-9 (Matrix Metalloproteinase 9), the presence of an amino acid change in said domain being indicative of susceptibility to said heart condition, post myocardial infarction is described, together with methods for drug design.

This application claims priority from U.S. provisional application No. 60/884,979 which is hereby incorporated by reference.

### FIELD OF THE INVENTION

The present invention relates to the use of a diagnostic marker and a platform for drug design in myocardial infarction and heart failure.

### BACKGROUND OF THE INVENTION

Congestive heart failure (CHF) is not a specific disease, but a compilation of signs and symptoms, all of which are caused by an inability of the heart to appropriately increase cardiac output as needed. Patients typically present with shortness of breath, edema and fatigue. CHF has become a disease of epidemic proportion, affecting 3% of the adult population. Mortality of CHF is worse than many forms of cancer with a five year survival of less than 30%. Myocardial infarction (MI) is one of the leading causes of CHF. Left ventricular remodelling contributes largely to CHF.

It is now well recognized that changes in the myocardial extracellular matrix (ECM) contribute to the progressive remodelling process. A balance of ECM synthesis and degradation, also called ECM turnover, determines the maintenance of tissue architecture. The normal rate of ECM synthesis in the heart is very low. During pathological situations, such as MI, collagen synthesis and deposition is accelerated not only in the infarcted, but also in the non-infarcted myocardium. Growing evidence suggests that changes in fibrillar collagen network and collagen matrix disorganization contribute to LV remodelling. Matrix disorganization has been attributed to increased expression of matrix metalloproteinases (MMPs), which break down matrix proteins and decrease expression of tissue inhibitors of metalloproteinase (TIMPs), a family of protease inhibitors. Polymorphonuclear leukocytes and macrophages are a rich source of MMPs. Recruitment and activation of monocytes/macrophages in the infarcted myocardium has been shown to contribute importantly to the processes that occur after MI.

Therefore, analogous to neurohormones and pro-inflammatory cytokines such as tumour necrosis factor-alpha (TNF-alpha), metalloproteinases (MMP) appear to represent another distinct class of biologically active molecules that can contribute to heart failure progression. There is a growing body of evidence that suggests that modulating inflammatory cytokines and MMP levels may represent a new therapeutic paradigm for treating patients with heart failure.

Among the MMP family containing so far 25 members, MMP-9 appears to be a key protein involved in ventricular remodelling. It has recently been shown that MMP activity is detrimental during the early phase following MI but beneficial during the late phase post MI, suggesting the existence of a therapeutic window that has to be carefully taken into account when applying therapeutic strategies to prevent or treat remodelling. Numerous studies have suggested the necessity to use specific MMP-9 inhibitors, since other members of the MMP family can have beneficial roles in ventricular remodelling. The same problematic scenario, i.e. inhibiting MMP-9 but not other MMPs, is encountered when one aims at treating other pathologies in which matrix degradation is involved. Several pharmaceutical companies have tested the possibility of creating such MMP-9-specific inhibitors, but early clinical trials have brought mixed results. Therefore, this highlights the need to unveil new strategies to develop specific MMP-9 inhibitors.

Single Nucleotide Polymorphisms (SNPs) in MMP-9 and TNF-alpha genes may act as susceptibility factors for the development of acute MI. In a preliminary study, we determined the presence of SNPs in the MMP-9 and TNF-alpha genes from circulating leukocytes. MassARRAY® technology was performed on a cohort of 98 patients with acute MI and 92 patients with atypical chest pain and normal coronary arteries. Among the 147 SNPs studied, nine of them showed a highly different frequency between both groups.

On the other hand, we conducted a clinical investigation in which we were able to show for the first time that MMP-9 plasma levels are correlated with the evolution of heart failure. In fact, MMP-9 levels are predictive of late onset CHF after MI. In our study, patients with low early MMP-9 levels had good late outcome: no CHF, normal ejection fraction and end-diastolic volume. However, patients with high early MMP-9 levels had a significant risk of late onset CHF (odds ratios of 6.5, p<0.006) and left ventricular remodelling (decreased ejection fraction, increased end-diastolic volume). Since our original publication from February 2006 (Wagner D R, Delagardelle C, Ernens I, Rouy D, Vaillant M, Beissel J. Matrix metalloproteinase-9 is a marker of heart failure after acute myocardial infarction. J Card Fail. 2006 February; 12(1): 66-72), our observations have been replicated by several other groups. Moreover, it was recently shown that MMP-9 levels decrease in patients who experience reverse remodelling and improvement of heart failure secondary to cardiac resynchronization therapy. Therefore, it is well recognized that plasma levels of MMP-9 may be predictive of ventricular remodelling and CHF following reperfused MI.

However, despite being able to assay MMP-9 levels in a patient, there is still a need in the art for predicting whether a patient is susceptible to post-MI heart failure, so that appropriate treatment can be prepared or patient education initiated.

### OBJECTS OF THE PRESENT INVENTION

The objects of the present invention are two-fold:

### 1. To Provide a Diagnostic Tool

It is an object of the present invention to provide a method for early diagnosis of the occurrence of congestive heart failure in order to improve survival and lessen the development of worsening heart failure.

It is another object of the present invention to use this diagnostic tool to identify susceptible patients at risk of developing ventricular remodelling and heart failure.

It is another object of the present invention to use this diagnostic tool to adjust treatments to better prevent the development of ventricular remodelling and heart failure after myocardial infarction.

### 2. To Provide a Platform for Drug Design

It is another object of the present invention to provide a platform for drug design to provide or identify molecule that can specifically inhibit MMP-9 activity.

It is another object of the present invention to decrease levels of active MMP-9 in a patient with myocardial infarction or heart failure.

It is a further object of the present invention to use this drug to treat patients after myocardial infarction in order to prevent maladaptive remodelling of the myocardium including fibrosis, apoptosis and necrosis.

It is another object of the present invention to use this drug to treat patients after myocardial infarction in order to prevent the development of heart failure. Furthermore, it is also an object of the present invention to administer therapeutically effective amounts of this drug to treat a patient with myocardial infarction, acute heart failure or chronic heart failure.

Surprisingly, we have discovered that a Single Nucleotide Polymorphism (SNP), present in the coding sequence of the MMP-9 gene, is found at different frequencies in patients with good or poor prognoses for heart failure following myocardial infarction. What is particularly surprising is that we have shown that the SNP leads to a single amino acid change in the hemopexin domain of the transcribed and active MMP-9 protein, resulting in an electrostatic change in the site on MMP-9 that binds TIMP-1. This is significant as TIMP-1 is the foremost inhibitor of MMP-9 activity.

### SUMMARY OF THE INVENTION

Thus, in a first aspect, the present invention provides a method for determining the susceptibility of an individual to a heart condition, post myocardial infarction, comprising detecting the presence of an amino acid change in the sequence of the hemopexin domain of MMP-9 (Matrix Metalloproteinase 9), the presence of an amino acid change in said domain being indicative of susceptibility to said heart condition, post myocardial infarction.

It is particularly preferred that the sequence that is detected comprises or encodes either an Arginine (Arg) or a Glutamine (Gln) amino acid residue at a position corresponding to position 148 of SEQ ID NOS. 6 or 8, or residue at a position corresponding to position 668 of SEQ ID NOS. 2 or 4. The presence of an Arg residue here is indicative of an individual at risk of suffering from or developing a heart condition post MI. The presence of a Gln residue here is indicative of a protective effect, i.e. an individual with a lower risk.

Preferably, the identity of a SNP (A/G) is detected at a position corresponding to position 443 of SEQ ID NOS 5 or 7, or at a position corresponding to position 7265 of SEQ ID NOS. 1 or 3. The presence of a Guanidine (G) nucleotide here is indicative of an individual at risk of suffering from or developing a heart condition post MI. The presence of an Adenine (A) nucleotide residue here is indicative of a protective effect, i.e. an individual with a lower risk.

The sequence of the hemopexin domain of MMP-9 (Matrix Metalloproteinase 9), also known as the PEX9 domain, is shown in (SEQ ID NOS. 5-8), of which SEQ ID NOS 6 and 8 are amino acid sequences and SEQ ID NOS 5 and 7 are polynucleotide sequences. The sequence of the domain may be detected by assessing the protein sequence per se or the nucleotide sequence encoding the protein domain. Due to the degeneracy of the genetic code, the nucleotide sequence (preferably DNA or RNA) may be any that encodes SEQ ID NOS. 6 or 8. Preferably, however, the nucleotide sequence is that according to SEQ ID NO. 5 or 7.

Preferably, the detected sequence is SEQ ID NO. 7, which is the DNA sequence of hemopexin domain of MMP-9 from the at risk (susceptible) group (showing a G nucleotide position 443).

Preferably, the detected sequence is SEQ ID NO. 8, which is the amino acid sequence of the hemopexin domain of MMP-9 from the at risk (susceptible) group (showing Arg at amino acid position 148).

It is also preferred that the detected sequence is SEQ ID NO. 5, which is the DNA sequence of the hemopexin domain of MMP-9 from the protective group (showing an Adenine nucleotide at position 443). Preferably, the detected sequence is SEQ ID NO. 6, which is the amino acid sequence of the hemopexin domain of MMP-9 from the protective group (showing a Gln amino acid residue at amino acid position 148).

Preferably, detecting the presence of an amino acid change in the sequence of the hemopexin domain is by comparing the nucleotide sequence of MMP-9 from the individual to SEQ ID NO. 1 or SEQ ID NO. 3.

It is particularly preferred that this detection may be by assessing the presence of at least one nucleotide at a position corresponding to position 7265 of SEQ ID NO. 1 or SEQ ID NO. 3. As mentioned above, the presence of a Guanidine nucleotide at this position, or one corresponding to it, is indicative of an individual susceptible to a heart condition, post myocardial infarction. Preferably, therefore, the individual has the MMP-9 sequence shown in SEQ ID NO. 3, comprising a Guanidine at said position.

Alternatively, this detection may be by observing the presence of Adenine at a position corresponding to position 7265 of SEQ ID NO. 1, an Adenine nucleotide at this position, or one corresponding to it, being protective against a heart condition, post myocardial infarction. In other words, an Adenine nucleotide at said position is indicative of an individual who is unlikely to be susceptible to said heart condition, post myocardial infarction. Preferably, the individual has the MMP-9 sequence shown in SEQ ID NO. 1, comprising an Adenine at said position.

Alternatively, or in addition, the detecting may be by detecting the presence of the amino acid change in said domain in the RNA transcribed from the gene. This is preferably mRNA.

More preferably, however, the detecting may be by detecting the presence of the amino acid change in said domain in the active or functional protein. Preferably the individual or patient has the MMP-9 protein according to SEQ ID NO. 2 comprising Glutamine (Gln) at position 668 of SEQ ID NO. 2, or one corresponding to it, being protective against heart failure, post myocardial infarction. Alternatively, the individual or patient has the MMP-9 protein according to SEQ ID NO. 4 comprising Arginine (Arg) at position 668 of SEQ ID NO. 2, or one corresponding to it, being indicative of susceptibility to heart failure, post myocardial infarction.

The individual or patient may be homozygous for the at risk or protective alleles or may be heterozygous.

Preferably, the heart condition is myocardial infarction, acute coronary syndrome, ischemic cardiomyopathy, non-ischemic cardiomyopathy or congestive heart failure. Most preferably, the heart condition is congestive heart failure.

The sampling of the protein sequence is preferably optionally in addition to the nucleotide analysis, i.e. both the genotype and the protein sequence of the active or functional protein are assessed (genomic and proteomic analysis).

The change in the amino acid sequence is preferably the result of the SNP referred to herein as SNP7265.

The present SNP involves a change from the more common Guanidine nucleotide to the less common Adenine nucleotide at a position corresponding to 7265 of SEQ ID NOS 1 or 3, as described above. This change results in an amino acid change from an Arginine amino acid residue to a Glutamine amino acid residue at a position corresponding to 668 of SEQ ID NOS 2 and 4, as also described above.

Preferably, the invention comprises assessing or determining the presence of the at risk allele, being the presence of G at the relevant position. Alternatively, or in addition, the invention preferably comprises assessing or determining the presence of the protective allele, being the presence of A at said position.

Where reference is made to SEQ ID NOS 1 and/or 3, this will be understood to include reference to the hemopexin domain sequences (SEQ ID NOS 5 and/or 7) and the corresponding numbering of the nucleotides therein, unless otherwise apparent. Equally, where reference is made to SEQ ID NOS 2 and/or 4, this will be understood to include reference to the hemopexin domain sequences (SEQ ID NOS 6 and/or 8) and the corresponding numbering of the amino acids therein, unless otherwise apparent.

The hemopexin domain of MMP-9 is preferably that defined by SEQ ID NOS 5-8, as discussed above, with or without the amino acid change brought about by the present SNP. The hemopexin domain is preferably that associated with TIMP binding, most preferably TIMP-1 binding.

The amino acid change resulting from the present SNP is strategically positioned in the hemopexin-like domain of MMP-9 (see FIGS. 1-3) and is therefore highly likely to be involved in binding not only to collagen fibres but also to the MMP-9 main inhibitor, Tissue Inhibitor of Metalloproteinase-1 (TIMP-1). Therefore, this SNP and the resulting amino acid change very likely plays a key role in ventricular remodelling and development of, or progression to, a heart condition, especially heart failure. This is borne out by the present Examples which show a statistically significant correlation between the presence of the protective allele and a high EF, low early phase MMP-9 activity (post MI), together with normal EF and EDV, with very low chances of developing heart failure, particularly Congestive Heart Failure (CHF) or Left Ventricle (LV) remodelling.

The window for the early phase activity of MMP-9 is preferably 1-2 days post MI and preferably less than 24 hours post MI. Therefore, to assess MMP-9 activity levels, it is preferred that MMP-9 is measured in plasma samples harvested up to 24 hours after MI.

The protein sequence assessed is preferably after any post-translation modifications.

Preferably the patient has already suffered a myocardial infarction. However, it is also envisaged that the patient may not yet have suffered a myocardial infarction, in which case the present SNP or the resulting amino acid change would be indicative of the likelihood of developing a heart condition after a Myocardial Infarction, should the individual then have a Myocardial Infarction. This is particularly useful for assessing a patient who might be at risk of an MI, for instance due to a family history thereof, having weak heart or other risk factor, and/or prior to an operation that may cause a Myocardial Infarction.

Thus, the present invention provides a screening method for infarcted patients, comprising determining the presence or absence of the above amino acid change. Similarly, the present invention also provides a screening method for the general population, infarcted or otherwise.

Also provided is a method of establishing a diagnosis and/or a prognosis in patients with myocardial infarction, by correlating an amino acid change on matrix-metalloproteinase-9 (MMP-9) with lower levels of MMP-9 activity, which indicates a better clinical outcome after myocardial infarction.

Thus, it is preferred that the measurable activity of MMP-9 is reduced, most preferably by a reduced in MMP-9 protein expression. This may be by down-regulation of expression of the gene or this may by competition or inhibition (such as stearic effects from inhibitor binding at the active site or elsewhere). The evidence to date is that there is a reduction in expression of MMP-9, hence a reduced measurable activity, resulting from the SNP.

The activity is measurable by a number of methods known in the art for proteases, which may for instance assay for the cleavage of a fluorescent marker. Zymography and/or ELISA are particularly preferred.

As shown herein, the activity of MMP-9 is reduced when the hemopexin domain is altered, most preferably by altering the sequence of domain, particularly the change resulting from the present SNP. This is particularly the case in the early phase window post MI, as defined above.

Following on from our previous studies, we investigated the genetic polymorphism of MMP-9 and its consequences on heart failure. For this purpose, we initiated sequencing experiments aiming at defining the presence or absence of SNPs in the entire sequence of the MMP-9 gene. Two groups of 22 patients with extreme phenotypes were selected: one group having a good clinical outcome after MI and absence of left ventricular remodelling and signs of heart failure (characterized by an ejection fraction of the left ventricle higher than 55%) and one group having a bad clinical outcome after MI and presence of left ventricular remodelling and signs of heart failure (characterized by an ejection fraction of the left ventricle lower than 40%). Several SNPs were identified in these patients. Among those, one SNP, located at position 7265 of the coding sequence of the MMP-9 gene, appeared particularly interesting. Indeed, the frequency of SNP7265 was different between the two groups of patients. This suggested that SNP7265 may be associated with the occurrence of heart failure after MI and may be used as a prognostic tool.

We next sought to determine whether the association between SNP7265 and heart failure observed in a small sample of patients with MI (44 patients) could be verified in a larger cohort. We used the Luxembourg Acute Myocardial Infarction (LUCKY) registry, which is maintained in our laboratory, to perform genotyping experiments to detect the presence of SNP7265 in 229 patients. First, these experiments allowed us to show that SNP7265 is associated with plasma levels of MMP-9 in a statistically significant manner. Second, we were able to show that SNP7265 is indeed significantly associated with the ejection fraction and the development of heart failure post MI.

These experiments revealed the potential usefulness of SNP7265 as a diagnostic tool to predict the occurrence of heart failure following MI. Also, we postulate that SNP7265 may be the starting point to develop therapeutic strategies aiming at decreasing the deleterious MMP-9 activity in the heart of MI patients.

According to a further aspect of the present invention, there is provided a method of establishing a diagnosis and a prognosis in patients with myocardial infarction by correlating an amino acid change in the hemopexin domain of matrix-metalloproteinase-9 (MMP-9) with lower MMP-9 levels, which indicates a better clinical outcome after myocardial infarction.

The amino acid change may be a single nucleotide polymorphism (SNP) in MMP-9, preferably localized at position 7265 of the coding sequence of MMP-9. The SNP may be a Guanidine to Adenine nucleotide change. The SNP induces an Arginine to Glutamine amino acid change.

Preferably, the heart condition is myocardial infarction, acute coronary syndrome, ischemic cardiomyopathy, non-ischemic cardiomyopathy or congestive heart failure. Most preferably, the heart condition is congestive heart failure.

According to another aspect of the present invention, there is provided a method of treatment to inhibit ventricular remodelling and to treat and prevent heart failure by correlating an amino acid change in the hemopexin domain of matrix-metalloproteinase-9 (MMP-9) with lower MMP-9 levels, which indicates a better clinical outcome after myocardial infarction.

According to a further aspect of the present invention, there is provided a method of predicting the occurrence of ventricular remodelling in patients following myocardial infarction, by correlating an amino acid change in the hemopexin domain of matrix-metalloproteinase-9 (MMP-9) with lower MMP-9 levels, which indicates a better clinical outcome after myocardial infarction.

According to yet a further aspect of the present invention, there is provided a method of improving the therapeutic strategy of a patient following myocardial infarction, based on correlating an amino acid change in the hemopexin domain of matrix-metalloproteinase-9 (MMP-9), which in turn is correlated with lower MMP-9 levels indicates a better clinical outcome.

According to another aspect of the present invention, there is provided a platform for drug design to treat ventricular remodelling is based on correlating an amino acid change in the hemopexin domain of matrix-metalloproteinase-9 (MMP-9) is correlated with lower MMP-9 levels, which indicates a better clinical outcome after myocardial infarction.

According to yet a further aspect of the present invention, there is provided a platform for drug development to specifically inhibit Matrix Metalloproteinase-9 (MMP-9) activity, wherein an amino acid change on matrix-metalloproteinase-9 (MMP-9) is correlated with lower MMP-9 levels and indicates a better clinical outcome after myocardial infarction.

Preferably, this is in relation to using the conformational and electrostatic changes in the secondary or tertiary structure of the MMP-9 protein, caused by the SNP's amino acid change, to provide new agonists or antagonists/inhibitors for MMP-9, especially those capable of mimicking or binding to the “altered” TIMP-binding site.

Alternatively, the invention may be used to develop strategies to block MMP-9 activity or reinforce its interaction with TIMP-1.

Thus, the present invention provides methods of identifying agonists or antagonists/inhibitors for MMP-9, or molecules capable of blocking MMP-9 activity, or reinforcing its interaction with TIMP-1 or reducing the detectable activity or expression of MMP-9.

The invention also provides a method of identifying agonists or antagonists/inhibitors for MMP-9, or molecules capable of blocking MMP-9 activity, or reinforcing its interaction with TIMP-1 or reducing the detectable activity or expression of MMP-9, comprising designing a molecule or ligand that will interact with the changed hemopexin domain of matrix-metalloproteinase-9 (MMP-9).

According to another aspect of the present invention, there is provided a method to improve MMP-9 binding to its natural inhibitor Tissue Inhibitor of MMP-1 (TIMP-1), by correlating an amino acid change in the hemopexin domain of matrix-metalloproteinase-9 (MMP-9) with lower MMP-9 levels and indicates a better clinical outcome after myocardial infarction.

According to a further aspect of the present invention, there is provided a method of preventing degradation of myocardial tissue associated with end-stage heart disease comprises administering therapeutically effective amounts of drug, wherein an amino acid change in the hemopexin domain of matrix-metalloproteinase-9 (MMP-9) is correlated with lower MMP-9 levels and indicates a better clinical outcome after myocardial infarction.

In a still further aspect of the present invention, there is provided a method of treating a patient presenting symptoms of congestive heart failure comprising administering an agent which decreases the activity of MMP-9 in the myocardial tissue, wherein an amino acid change in the hemopexin domain of matrix-metalloproteinase-9 (IvIMP-9) is correlated with lower MMP-9 levels and indicates a better clinical outcome after myocardial infarction.

The symptoms may be indicative of chronic or acute heart failure and the agent which decreases the production of MMP-9 in the myocardial tissue may be comprised of a therapeutically effective drug.

For the avoidance of doubt, the amino acid change, in any aspect of the present invention may be a single nucleotide polymorphism (SNP) in MMP-9, preferably localized at position 7265 of the coding sequence of MMP-9. The SNP may be a Guanidine to Adenine nucleotide change. The SNP induces an Arginine to Glutamine amino acid change as defined further above. Preferably, the heart condition is myocardial infarction, acute coronary syndrome, ischemic cardiomyopathy, non-ischemic cardiomyopathy or congestive heart failure. Most preferably, the heart condition is congestive heart failure. The drug or agent may be administered orally, intravenously, intradermally, transdermally or expressed in a suitable vector.

Also provided is a method of determining susceptibility to a heart condition in an individual, comprising detecting an at-risk allele of a SNP, wherein the SNP is located within a sequence encoding the hemopexin domain of the active MMP-9 protein.

The invention also provides a method for assaying for the presence of a first polynucleotide having a SNP associated with susceptibility to a heart condition in a sample, comprising; contacting said sample with a second polynucleotide, wherein said second polynucleotide comprises a nucleotide sequence selected from the group consisting SEQ. ID. NOS.: 1,3, 5, 7, 9 and/or 10 and the complements of said sequences, wherein said second polynucleotide hybridizes to said first polynucleotide under stringent conditions. The stringent condition are preferably highly stringent, preferably 6×SSC.

Also provided is a vector comprising an isolated polynucleotide containing a SNP associated with susceptibility to a heart condition, wherein said isolated polynucleotide is operably linked to a regulatory sequence, preferably a suitable promoter. Also provided is a host cell comprising said vector.

In all aspects the SNP associated with susceptibility to a heart condition results in the present amino acid change in the hemopexin domain of the transcribed active MMP-9 protein, and most preferably SNP7265 as described herein, namely G to A nucleotide change resulting in an Arg to Gln amino acid change in said domain. The invention also provides a method for producing a polypeptide encoded by an isolated polynucleotide having a SNP associated with susceptibility to a heart condition; comprising culturing the above recombinant host cell under conditions suitable for expression of said polynucleotide.

Also provided is a method of assaying for the presence of a polypeptide encoded by an isolated polynucleotide having a SNP associated with susceptibility to a heart condition in a sample, said method comprising contacting the sample with an antibody which specifically binds to said encoded polypeptide.

The invention further provides a transgenic animal comprising a polynucleotide having a SNP associated with susceptibility to a heart condition.

Also provided is a method of identifying an agent that alters expression of a polynucleotide containing a SNP associated with susceptibility to a heart condition comprising:

(a) contacting a polynucleotide with an agent to be tested under conditions for expression, wherein the polynucleotide comprises (1) a SNP associated with susceptibility to a heart condition and (2) a promoter region operably linked to a reporter gene;
(b) assessing the level of expression of the reporter gene in the presence of the agent;
(c) assessing the level of expression of the reporter gene in the absence of the agent; and
(d) comparing the level of expression in step (b) with the level of expression in step (c) for differences which indicate that expression was altered by the agent.

The invention also provides a method for assaying a sample for the presence of a first polynucleotide which is at least partially complementary to a part of a second polynucleotide wherein the second polynucleotide comprises a sequence selected from the group consisting of sequences identified by SEQ. ID. NOS. 1,3, 5, 7, 9 and/or 10, and the complements thereof, comprising:

a) contacting said sample with said second polynucleotide under conditions appropriate for hybridization, and
b) assessing whether hybridization has occurred between said first and said second polynucleotide.
wherein if hybridization has occurred, said first polynucleotide is present in said sample. Suitable markers of hybridization are known in the art.

Also provided is a reagent for assaying a sample for the presence of a first polynucleotide comprising a SNP associated with susceptibility to a heart condition, said reagent comprising a second polynucleotide comprising a contiguous nucleotide sequence which is at least partially complementary to a part of the first polynucleotide.

The invention further provides a reagent kit for assaying a sample for the presence of a first polynucleotide comprising a SNP associated with susceptibility to a heart condition, comprising in separate containers:

a) one or more labelled second polynucleotides comprising a sequence selected from the group consisting of sequences identified by SEQ. ID. NOS.: 1,3, 5, 7, 9 and/or 10, and the complements thereof and
b) reagents for detection of said label.

Also provided is a method of diagnosing a susceptibility to a heart condition in an individual comprising detecting a haplotype associated with said condition, the haplotype comprising the present SNP and at least one other haplotype. Preferably said at least one further haplotype is also associated with post-MI disease states, particularly heart conditions.

Preferably detecting the presence of the haplotype comprises enzymatic amplification of nucleic acid from the individual. Preferably, detecting the presence of the haplotype further comprises electrophoretic analysis. Detecting the presence of the haplotype may further comprise restriction fragment length polymorphism analysis. Detecting the presence of the haplotype may further comprise sequence analysis.

Also provided is a method of diagnosing susceptibility to a heart condition in an individual, comprising:

a) obtaining a polynucleotide sample from said individual; and
b) analyzing the polynucleotide sample for the presence or absence of a haplotype, wherein the presence of the haplotype corresponds to a susceptibility to said heart condition.

Also provided is a method of identifying a gene associated with susceptibility to a heart condition comprising: (a) identifying a gene containing a SNP that is located within a sequence selected from the group consisting of sequences identified by SEQ. ID. NOS. 1,3, 5, 7, 9 and/or 10, and the complements thereof and (b) comparing the expression of said gene in an individual having an at-risk allele with the expression of said gene in an individual not having an at-risk allele for differences indicating that the gene is associated with susceptibility to said heart condition.

The invention further provides a method of identifying an agent suitable for treating a heart condition; comprising: (a) contacting a polynucleotide with an agent to be tested, wherein the polynucleotide contains a SNP located within a sequence selected from the group consisting of sequences identified by SEQ. ID. NOS. 1,3, 5, 7, 9 and/or 10, and the complements thereof; and (b) determining whether said agent binds to, alters, or affects the polynucleotide in a manner which would be useful for treating said condition.

Yet further provided is a method of identifying an agent suitable for treating a heart condition comprising:

(a) contacting a polypeptide with an agent to be tested, wherein the polypeptide is SEQ ID NO 2 or 4 or is encoded by a polynucleotide containing a SNP located within a sequence selected from the group consisting of sequences identified by SEQ. ID. NOS. 1,3, 5, 7, 9 and/or 10, and the complements thereof; and (b) determining whether said agent binds to, alters, or affects the polypeptide in a manner which would be useful for treating said heart condition.

Also provided is a drug or agent identified by said methods, a pharmaceutical composition containing said agent in a therapeutically effective amount.

### BRIEF DESCRIPTION OF THE DRAWINGS

FIG. 1 is an illustration of the 3D structure of the hemopexin domain of MMP-9 highlighting the position of arginine.

FIG. 2 shows a 3D structure of human MMP-2 hemopexin domain containing a glutamine at the same (i.e. a corresponding) position.

FIG. 3A illustrates a 3D structure prediction of the MMP-9 structure without mutation.

FIG. 3B illustrates a 3D structure prediction of the MMP-9 structure with mutation.

FIG. 4A shows a statistically significant association between SNP7275 and the level of plasma MMP-9 in patients following acute MI.

FIG. 4B shows a statistically significant association between SNP7275 and the ejection fraction of patients four months after MI.

FIG. 5 provides the full genomic DNA sequence and amino acid sequence of human MMP-9. Highlighted is the SNP at location 7265 of the nucleotide sequence (exon 12) that induces—when converted from a guanidine (symbol “G”) to an adenine (symbol “A”)—a change in the amino acid arginine (symbol “R”) to glutamine (symbol “Q”), which is also highlighted by bold and underlined characters in the amino acid sequence (amino acid 668).

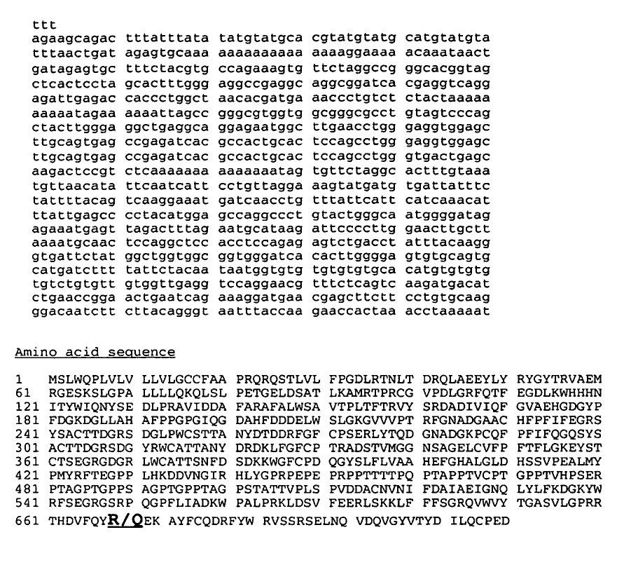

### DETAILED DESCRIPTION

Matrix metalloproteinases (MMPs) are very important compounds that are the driving force behind the degradation of the myocardial extracellular matrix. Recent studies have clearly demonstrated that in the heart, MMPs contribute to ventricular remodelling and heart failure. At the clinical level, studies from our group recently confirmed by others have shown that elevated blood levels of MMPs are associated with the development of heart failure after MI. Therefore, measurement of MMPs, and in particular MMP-9, in patients with MI or CHF provides a prognostic measure similar to that of TNF-alpha, angiotensin II or norepinephrine. Neutrophils and macrophages play an important role in the inflammatory responses that lead to myocardial damage and fibrosis, at least partly through production of large quantities of MMP-9.

We investigated the mechanisms responsible for regulation of MMP-9 activity during remodelling, and we tested the hypothesis that genetic polymorphisms of the MMP-9 gene could modify MMP-9 activity.

For this purpose, we first selected 44 patients with myocardial infarction, 22 of them with a favourable outcome (EF>55%) and 22 with an unfavourable outcome (EF<40%). Genomic DNA was extracted from peripheral blood mononuclear cells isolated from the venous blood of these patients. The whole MMP-9 gene (9 kBytes) was sequenced. In parallel, MMP-9 plasma concentrations were determined with gelatin zymography.

We identified 5 SNPs which vary significantly between the two populations of patients. Among these, one SNP at position 7265 of the coding sequence of the MMP-9 gene (called SNP7265) is strongly associated with modifications of the structure and activity of MMP-9. This SNP induces a change in the amino acid composition of the hemopexin domain of MMP-9, the same domain where TIMP-1 interacts with MMP-9. TIMP-1 is the most important natural brake of MMP-9. The mere existence of this SNP was already known. However, its clinical and therapeutic importance was totally unrecognized.

We then verified the significance of SNP7265 in a larger cohort of patients with acute MI (229 patients). We were able to reveal a statistically significant association between SNP7265, plasma MMP-9 levels and ejection fraction. The presence of the mutation improves the clinical outcome of the MI patients since it is associated with lower plasma MMP-9 levels and higher ejection fraction. The mutation is therefore protective and reduces the chances of developing heart failure after MI.

Therefore, we propose a new method to use this SNP in two complementary ways: first, as a diagnostic and prognostic method to identify patients at risk to develop heart failure after myocardial infarction and second, as a platform for drug design aimed to specifically inhibit MMP-9 activity.

Our original publication on MMP-9 was published in February 2006 (Wagner et al, supra) and showed that Matrix Metalloproteinase-9 (MMP-9) is a marker of heart failure after acute myocardial infarction. However, this paper is completely silent on SNPs or nucleotide or amino acid changes that could be protective against, or indicative of susceptibility to, a heart condition, post MI.

Although the mere existence of this SNP (referred to herein as SNP 7265) was known, no function has ever been associated with it. This is because the SNP was identified (and provided with the ref SNP ID rs2274756), along with many other SNPs, by several research groups that sequenced the genomes of hundreds of people from different populations. These individuals had not been associated with a disease state, so there was never any comparison of the frequency of SNP between two or more groups of patients (i.e. normal vs. diseased; or sick vs. not sick). In other words, the SNP was identified as part of a genome sequencing project and has never been linked with the incidence of a heart condition post MI.

Also provided is a method to improve MMP-9 binding to its natural inhibitor Tissue Inhibitor of MMP-1 (TIMP-1), a method of preventing degradation of myocardial tissue associated with end-stage heart disease comprising administering therapeutically effective amounts of drug, and a method of treating a patient presenting symptoms of congestive heart failure comprising administering an agent which decreases the activity of MMP-9 in the myocardial tissue.

Where reference to the SNP is made, it will be understood that this encompasses both the nucleotide change and the resulting amino acid change, unless otherwise apparent.

A single nucleotide polymorphism, or SNP, is generally accepted to be a DNA sequence variation occurring when a single nucleotide—A, T, C, or G—in the genome differs between members of a species (or between paired chromosomes in an individual). For example, two sequenced DNA fragments from different individuals, AAGCCTA to AAGCTTA, contain a difference in a single nucleotide. In this example there are said to be two alleles: C and T.

In the present invention, there are also two alleles: the protective allele (comprising Adenine and encoding Gln) and the risk/susceptible allele (comprising Guanidine and encoding Arg).

It will also be appreciated the whole codon encoding Gln (CAA or CAG) may be detected. Alternatively, or in addition, the whole codon encoding Arg (CGA or CGG) may be detected. Optionally, addition flanking nucleotides may also be detected, preferably at least 2, more preferably at least 5, more preferably at least 10, more preferably at least 15, more preferably at least 20 and, more preferably at least 25. The number on either side of the SNP nucleotide or codon may be the same or different.

SNPs are often found to be the etiology of many human diseases and are of particular interest in pharmacogenetics. Thus, pharmacogenetic analysis and tailoring of treatment and medication to each individual are also encompassed within the present invention.

The present SNP can also provide a genetic fingerprint for use in identity testing.

The detection step may involve isolating the protein or polynucleotide sequence and determining the identity of the relevant nucleotide or nucleotides or amino acid residue or residues.

Suitable methods for detecting the SNP, will be known to the skilled person, but may include any of those described below. The invention is not limited to any of such methods.

Suitable methods may include hybridization-based methods, including Dynamic Allele-Specific Hybridization (DASH), which takes advantage of the differences in the melting temperature in DNA that results from the instability of mismatched base pairs. The process can be vastly automated and encompasses a few simple principles. In the first step, a genomic segment is amplified and attached to a bead through a PCR reaction with a biotinylated primer. In the second step, the amplified product is attached to a streptavidin column and washed with NaOH to remove the unbiotinylated strand. An allele specific oligonucleotide is then added in the presence of a molecule that fluoresces when bound to double-stranded DNA. The intensity is then measured as temperature is increased until the Tm can be determined. An SNP will result in a lower than expected Tm.

SNP detection through Molecular beacons makes use of a specifically engineered single-stranded oligonucleotide probe. The oligonucleotide is designed such that there are complementary regions at each end and a probe sequence located in between. This design allows the probe to take on a hairpin, or stem-loop, structure in its natural, isolated state. Attached to one end of the probe is a fluorophore and to the other end a fluorescence quencher. Because of the stem-loop structure of the probe, the fluorophore is in close proximity to the quencher, thus preventing the molecule from emitting any florescence. The molecule is also engineered such that only the probe sequence is complementary to the genomic DNA that will be used in the assay.

If the probe sequence of the molecular beacon encounters its target genomic DNA during the assay, it will anneal and hybridize. Because of the length of the probe sequence, the hairpin segment of the probe will denature in favour of forming a longer, more stable probe-target hybrid. This conformational change permits the fluorophore and quencher to be free of their tight proximity due to the hairpin association, allowing the molecule to fluoresce. If on the other hand, the probe sequence encounters a target sequence with as little as one non-complementary nucleotide, the molecular beacon will preferentially stay in its natural hairpin state and no fluorescence will be observed, as the fluorophore remains quenched.

The unique design of these molecular beacons allows for a simple diagnostic assay to identify SNPs at a given location. If a molecular beacon is designed to match a wild-type allele and another to match a mutant of the allele, the two can be used to identify the genotype of an individual. If only the first probe's fluorophore wavelength is detected during the assay then the individual is homozygous to the wild type. If only the second probe's wavelength is detected then the individual is homozygous to the mutant allele. Finally, if both wavelengths are detected, then both molecular beacons must be hybridizing to their complements and thus the individual must contain both alleles and be heterozygous.

SNP microarrays may also be used. In high density oligonucleotide SNP arrays, hundreds of thousands of probes are arrayed on a small chip, allowing for a large number of SNPs to be interrogated simultaneously, thus allowing for other risk factors to also be detected, in addition to the present invention.

Enzyme-based methods, including DNA ligase, DNA polymerase and nucleases may also be employed, as these are considered to be high-fidelity SNP genotyping methods.

Restriction fragment length polymorphism (RFLP) is considered to be one of the simplest and earliest methods of detecting SNPs. SNP-RFLP makes use of the many different restriction endonucleases and their high affinity to unique and specific restriction sites. By performing a digestion on a genomic sample and determining fragment lengths through a gel assay it is possible to ascertain whether or not the enzymes cut the expected restriction sites. A failure to cut the genomic sample results in an identifiably larger than expected fragment implying that there is a mutation at the point of the restriction site which is rendering it protected from nuclease activity.

PCR-based methods are also envisaged. For instance, Tetra-primer ARMS-PCR employs two pairs of primers to amplify two alleles in one PCR reaction. The primers are designed such that the two primer pairs overlap at a SNP location but each match perfectly to only one of the possible SNPs. As a result, if a given allele is present in the PCR reaction, the primer pair specific to that allele will produce product but not to the alternative allele with a different SNP. The two primer pairs are also designed such that their PCR products are of a significantly different length allowing for easily distinguishable bands by gel electrophoresis.

In examining the results, if a genomic sample is homozygous, then the PCR products that result will be from the primer which matches the SNP location to the outer, opposite strand primer as well from the two opposite, outer primers. If the genomic sample is heterozygous, then products will result from the primer of each allele to their respective outer primer counterparts as well as from the two opposite, outer primes.

Flap endonuclease (FEN) is an endonuclease that catalyzes structure-specific cleavage. This cleavage is highly sensitive to mismatches and can be used to interrogate SNPs with a high degree of specificity.

In the basic Invader assay, a FEN called cleavase is combined with two specific oligonucleotide probes, that together with the target DNA, can form a tripartite structure recognized by cleavase. The first probe, called the Invader oligonucleotide is complementary to the 3′ end of the target DNA. The last base of the Invader oligonucleotide is a non-matching base that overlaps the SNP nucleotide in the target DNA. The second probe is an allele-specific probe which is complementary to the 5′ end of the target DNA, but also extends past the 3′ side of the SNP nucleotide. The allele-specific probe will contain a base complementary to the SNP nucleotide. If the target DNA contains the desired allele, the Invader and allele-specific probes will bind to the target DNA forming the tripartite structure. This structure is recognized by cleavase, which will cleave and release the 3′ end of the allele-specific probe. If the SNP nucleotide in the target DNA is not complementary allele-specific probe, the correct tripartite structure is not formed and no cleavage occurs. The Invader assay is usually coupled with fluorescence resonance energy transfer (FRET) system to detect the cleavage event. In this setup, a quencher molecule is attached to the 3′ end and a fluorophore is attached to the 5′ end of the allele-specific probe. If cleavage occurs, the fluorophore will be separated from the quencher molecule generating a detectable signal.

Only minimal cleavage occurs with mismatched probes making the Invader assay highly specific. However, in its original format, only one SNP allele could be interrogated per reaction sample and it required a large amount of target DNA to generate a detectable signal in a reasonable time frame. Several developments have extended the original Invader assay. By carrying out secondary FEN cleavage reactions, the Serial Invasive Signal Amplification Reaction (SISAR) allows both SNP alleles to be interrogated in a single reaction. SISAR Invader assay also requires less target DNA, improving the sensitivity of the original Invader assay. The assay has also been adapted in several ways for use in a high-throughput format. In one platform, the allele-specific probes are anchored to microspheres. When cleavage by FEN generates a detectable fluorescent signal, the signal is measured using flow-cytometry. The sensitivity of flow-cytometry, eliminates the need for PCR amplification of the target DNA. These high-throughput platforms have not progressed beyond the proof-of-principle stage and so far the Invader system has not been used in any large scale SNP genotyping projects.

Other suitable methods include Primer extension analysis, 5′-nuclease assays (such as the Taqman® assay for SNP genotyping), an oligonucleotide ligase assay, single strand conformation polymorphism assays, Temperature Gradient Gel Electrophoresis (TGGE), Denaturing high performance liquid chromatography (DHPLC), High-Resolution Melting of the entire amplicon, SNPlex (a proprietary genotyping platform sold by Applied Biosystems) or even by sequencing (especially by pyrosequencing).

The term “SNP” refers to a single nucleotide polymorphism at a particular position in the human genome that varies among a population of individuals. As used herein, a SNP maybe identified by its name or by location within a particular sequence.

As used herein, the nucleotide sequences disclosed by the SEQ. ID. NOS. 1,3,5,7, 9 and 10 of the present invention encompass the complements of said nucleotide sequences. In addition, as used herein, the term “SNP” encompasses any allele among a set of alleles.

The term “allele” refers to a specific nucleotide among a selection of nucleotides defining a SNP. The term “minor allele” refers to an allele of a SNP that occurs less frequently within a population of individuals than the major allele, for instance the protective allele of the present invention (comprising A). The term “major allele” refers to an allele of a SNP that occurs more frequently within a population of individuals than the minor allele, for instance the at risk allele of the present invention (comprising G).

The term “at-risk allele” refers to an allele that is associated with susceptibility to a heart condition post MI. The term “haplotype” refers to a combination of particular alleles from two or more SNPs.

The term “polynucleotide” refers to polymeric forms of nucleotides of any length. The polynucleotides may contain deoxyribonucleotides, ribonucleotides, and/or their analogues. Polynucleotides may have any three-dimensional structure including single-stranded, double-stranded and triple helical molecular structures, and may perform any function, known or unknown. The following are non-limiting embodiments of polynucleotides: a gene or gene fragment, exons, introns, mRNA, tRNA, rRNA, short interfering nucleic acid molecules (siRNA), ribozymes, cDNA, recombinant polynucleotides, branched polynucleotides, plasmids, vectors, isolated DNA of any sequence, isolated RNA of any sequence, nucleic acid probes, and primers. A polynucleotide may also comprise modified nucleic acid molecules, such as methylated nucleic acid molecules and nucleic acid molecule analogues.

A “substantially isolated” or “isolated” polynucleotide is one that is substantially free of the sequences with which it is associated in nature. By substantially free is meant at least 50%, at least 70%, at least 80%, or at least 90% free of the materials with which it is associated in nature. An “isolated polynucleotide” also includes recombinant polynucleotides, which, by virtue of origin or manipulation: (1) are not associated with all or a portion of a polynucleotide with which it is associated in nature, (2) are linked to a polynucleotide other than that to which it is linked in nature, or (3) does not occur in nature.

The term “hybridizes under stringent conditions” is intended to describe conditions for hybridization and washing under which nucleotide sequences at least 60%, 65%, 70%, 75%, 80%, 85%, 90%, 95%, or 98% identical to each other typically remain hybridized to-each other. Such stringent conditions are known to those skilled in the art and can be found in Current Protocols in Molecular Biology, John Wiley & Sons, N.Y (1989), 6.3.1-6.3.6. A non-limiting example of stringent hybridization conditions are hybridization in 6× sodium chloride/sodium citrate (SSC) at about 45 degrees C., followed by one or more washes in 0.2.×SSC, 0.1% SDS at 50-65 degrees C.

Where reference to a particular sequence, amino acid or nucleotide, is made, then it will be appreciated that this includes preferably at least 70% sequence homology to said reference sequence and more preferably at least 75%, more preferably at least 80%, more preferably at least 85%, more preferably at least 90%, more preferably at least 95%, more preferably at least 99%, more preferably at least 99.5%, and even more preferably at least 99.9% homology, unless otherwise apparent. Suitable methods for establishing this include the BLAST program.

The term “vector” refers to a DNA molecule that can carry inserted DNA and be perpetuated in a host cell. Vectors are also known as cloning vectors, cloning vehicles or vehicles. The term “vector” includes vectors that function primarily for insertion of a nucleic acid molecule into a cell, replication vectors that function primarily for the replication of nucleic acids, and expression vectors that function for transcription and/or translation of the DNA or RNA. Also included are vectors that provide more than one of the above functions.

A “host cell” includes an individual cell or cell culture which can be or has been a recipient for vector(s) or for incorporation of nucleic acid molecules and/or proteins. Host cells include progeny of a single host cell, and the progeny may not necessarily be completely identical (in morphology or in total DNA complement) to the original parent due to natural, accidental, or deliberate mutation. A host cell includes cells transfected with the polynucleotides of the present invention. An “isolated host cell” is one which has been physically dissociated from the organism from which it was derived.

The terms “individual,” “host,” and “subject” are used interchangeably herein to refer to a vertebrate, preferably a mammal, more preferably a human.

The terms “transformation,” “transfection,” and “genetic transformation” are used interchangeably herein to refer to the insertion or introduction of an exogenous polynucleotide into a host cell, irrespective of the method used for the insertion, for example, lipofection, transduction, infection, electroporation, CaPU4 precipitation, DEAE-dextran, particle bombardment, etc. The exogenous polynucleotide may be maintained as a non-integrated vector, for example, a plasmid, or alternatively, may be integrated into the host cell genome. The genetic transformation may be transient or stable.

The present invention employs, unless otherwise indicated, conventional techniques of molecular biology (including recombinant techniques), microbiology, cell biology, biochemistry and immunology, which are within the skill of the art.

As used herein, the singular form of any term can alternatively encompass the plural form and vice versa. All publications and references cited herein are incorporated by reference in their entirety for any purpose.

The present invention will now be described in relation to the following non-limiting Example.

### Example

### Patients and Methods

To increase our chances to detect relevant SNPs in the context of ventricular remodelling, we selected two groups of patients having “extreme” phenotypes after myocardial infarction, namely patients that evolved very favourably after infarction (EF>55%, average 60%) and patients that evolved unfavourably (EF<40%, average 29%). Each group contained 22 patients.

Genomic DNA was extracted from peripheral blood mononuclear cells following Ficoll separation. Extraction was performed with the FlexiGene DNA kit (Qiagen) according to the manufacturer's instructions. DNA quantity and quality was assessed using a Nanodrop spectrophotometer. DNA integrity was assessed through agarose gel electrophoresis.

The full MMP-9 gene was sequenced in all patients, including promoter, coding sequence and untranslated regions (total 9 kb), without knowing the patient's phenotype.

Plasma levels of MMP-9 were determined by gelatin zymography.

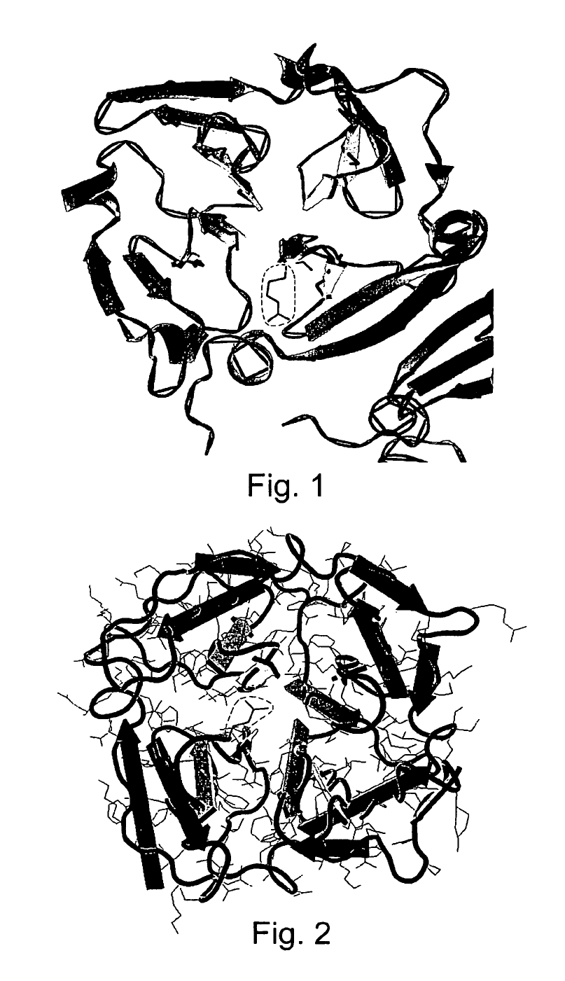

Referring now to FIG. 1, the hemopexin domain of MMP-9 with the position of arginine highlighted (encircled by a dashed line). Its net positive charge should be involved in the MMP-9/TIMP-1 interaction. A putative modification can be expected if arginine is replaced by glutamine, a negative polar amino acid.

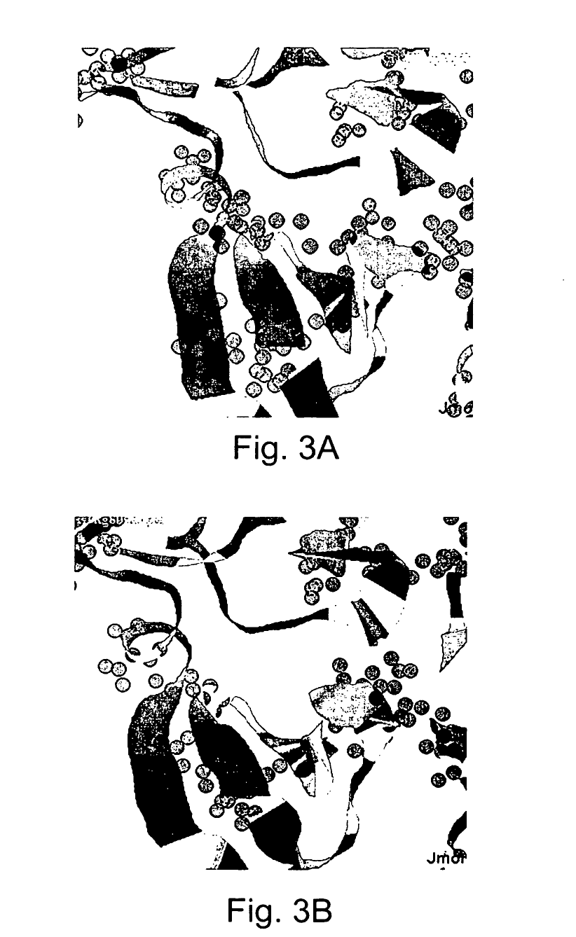

As shown in FIG. 2, human MMP-2 hemopexin domain contains a glutamine at the same position. A 3D structure shows that this amino acid (encircled by a dashed line) is likely to be involved in interactions with inhibitors.

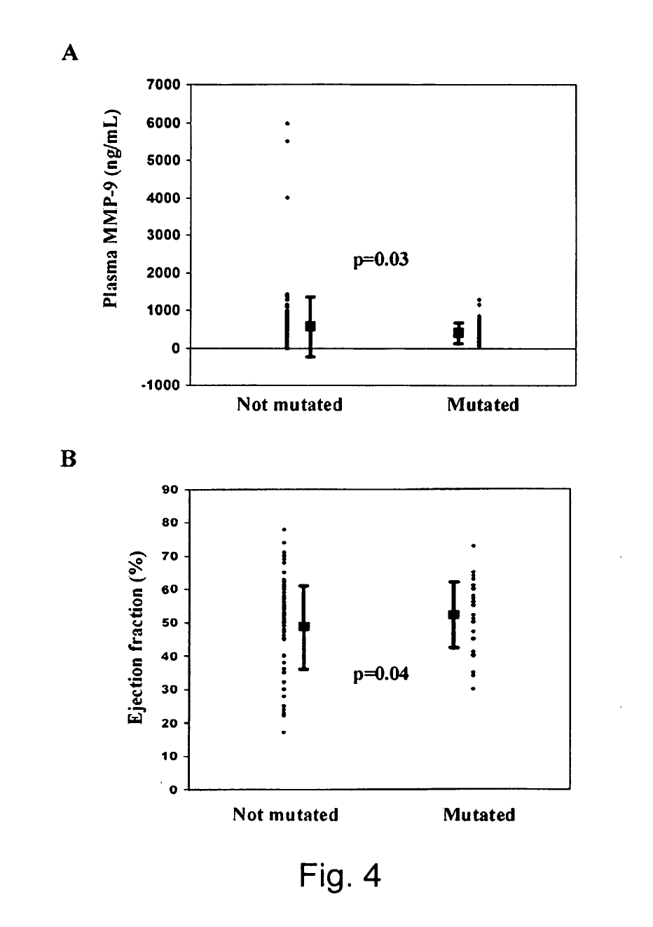

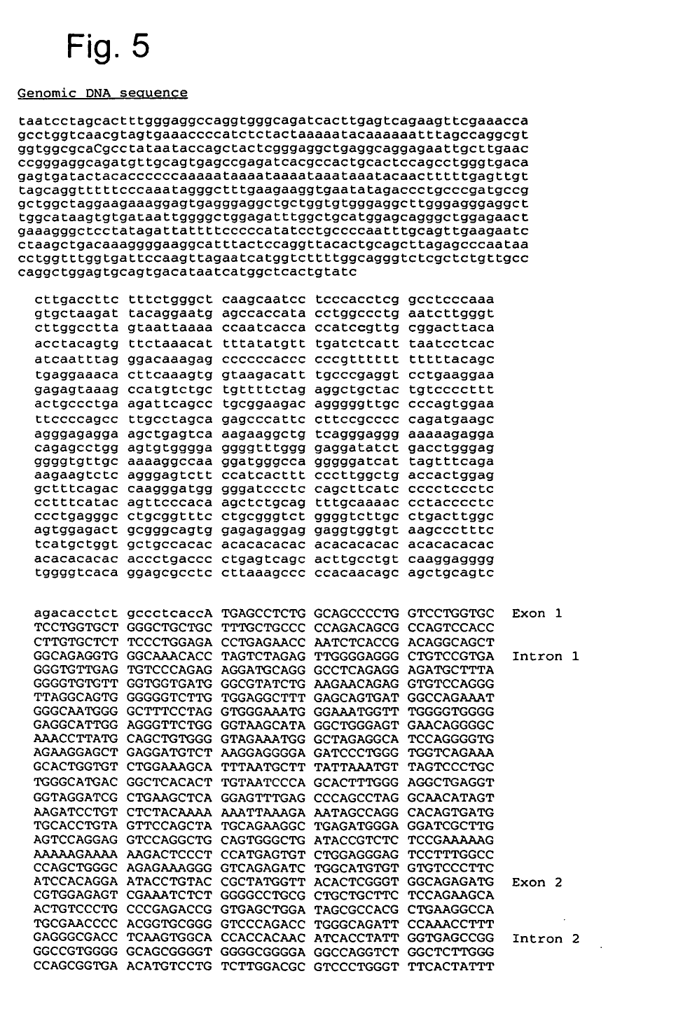

A prediction of the MMP-9 structure with the MAGOS software is shown in FIGS. 3A and 3B. Highlighted (by light coloured spots) are the positively charged amino acids.

We clearly show a change in the polarity of the protein when the SNP changes the amino acid glutamine (FIG. 3B) to arginine (FIG. 3A).

Additional genotyping experiments to detect the presence of SNP7265 were performed on a larger cohort from the Luxembourg Acute Myocardial Infarction (LUCKY) registry. This registry has been accepted by the local ethics committee and data protection committee. 229 patients were genotyped using the Taqman® technology and the following probe:

GACACGCACGACGTCTTCCAGTACC[A/G]AGGTGAGG
GCTGAGGAGGATCCCTT.
(SEQ ID NOS 9 (comprising A at position 26 and
SEQ ID NO. 10 comprising G at position 26))

All patients had documented plasma levels of MMP-9 (measured by Enzyme-Linked ImmunoSorbent Assay, ELISA) and 125 patients had documented ejection fraction (measured by echocardiography) 4 months following MI.

### Results

1. Plasma levels of MMP-9 were 660 pixels' in the low EF group, and 437 pixels' in the high EF group. This was in accordance with our previous work showing that MMP-9 is a predictor of EF after myocardial infarction.

2. Five SNPs were identified with different frequencies between both groups. Among these, one SNP, localized in position 7265 of the coding sequence shows a known SNP (rs2274756) with a Guanidine to Adenine nucleotide change engendering an Arginine to Glutamine amino acid change. This change was present in 2 patients in the low EF group and 6 patients in the high EF group. Average MMP-9 levels in A/G heterozygous patients were 215 pixels', as compared to 675 pixels' in homozygous G/G patients (p=0.004). This amino acid change is therefore correlated with lower MMP-9 levels and a better clinical outcome after MI (6 patients with EF of 60% vs. 2 patients with EF of 35%).

3. Genotyping experiments on patients with acute MI revealed a statistically significant association between the presence of SNP7265 and MMP-9 plasma levels on one hand and between SNP7265 and the ejection fraction on the other hand.

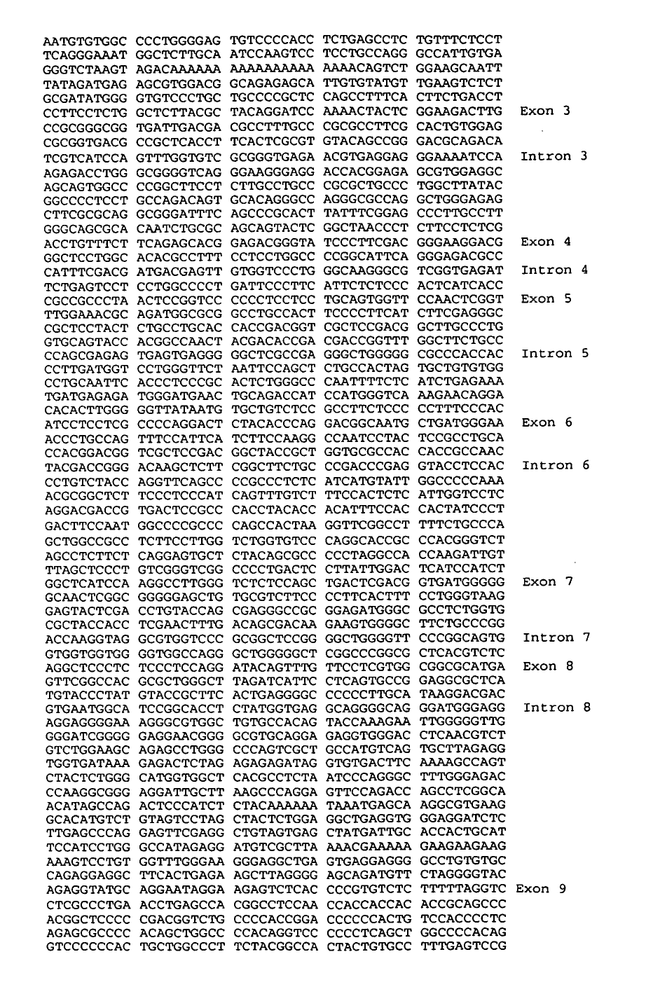

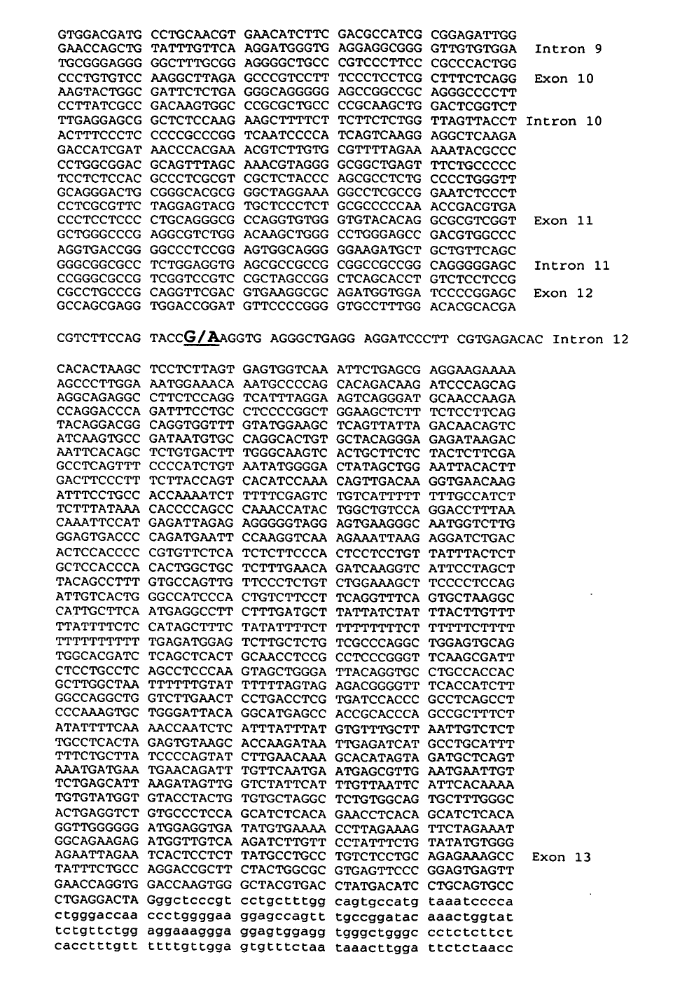

Mean values for plasma MMP-9 were 562.41 ng/mL in the non mutated patients (n=176) vs. 404.55 ng/mL in the mutated patients (n=53) (P=0.03). See FIG. 4A. This indicates that the presence of SNP7265 is associated with lower plasma MMP-9 levels.

Mean values for ejection fraction were 48.65% in the non mutated patients (n=93) vs. 52.23% in the mutated patients (n=32) (P=0.04). See FIG. 4B. This indicates that the presence of SNP7265 is associated with higher ejection fraction and therefore better clinical outcome after MI.

Due to its strategic position in the hemopexin-like domain of MMP-9 (see drawings below) involved in binding not only to collagen fibres but also to the MMP-9 main inhibitor, Tissue Inhibitor of Metalloproteinase-1 (TIMP-1), this SNP is very likely to play a key role in ventricular remodelling and heart failure development.

### BRIEF DESCRIPTION OF THE SEQUENCES

SEQ ID NO. 1: DNA sequence of MMP-9 from protective group—Adenine (A) at position 7265.
SEQ ID NO. 2 Amino acid sequence of MMP-9 from protective group—Gln (Q) at position 668.
SEQ ID NO. 3: DNA sequence of MMP-9 from at risk (susceptible) group—Guanidine (G) at position 7265.
SEQ ID NO. 4 Amino acid sequence of MMP-9 from at risk (susceptible) group—Arg (R) at position 668.
SEQ ID NO. 5: DNA sequence of hemopexin domain of MMP-9 from protective group—Adenine (A) at position 443.
SEQ ID NO. 6: Amino acid sequence of hemopexin domain of MMP-9 from protective group—Gin (Q) at position 148.
SEQ ID NO. 7 DNA sequence of hemopexin domain of MMP-9 from at risk (susceptible) group—Guanidine (G) at position 443).
SEQ ID NO. 8 Amino acid sequence of hemopexin domain of MMP-9 from at risk (susceptible) group—Arginine (R) at position 148).
SEQ ID NO. 9: probe comprising A at position 26.
SEQ ID NO. 10: probe comprising G at position 26.

1. A method for determining the susceptibility of an individual to a heart condition, post myocardial infarction, comprising detecting the presence of an amino acid change in the sequence of the hemopexin domain of MMP-9 (Matrix Metalloproteinase 9), the presence of an amino acid change in said domain being indicative of susceptibility to said heart condition, post myocardial infarction.

2. A method according to claim 1, wherein the sequence that is detected comprises or encodes either a Glutamine (Gln) or an Arginine (Arg) amino acid residue at a position corresponding to position 148 of SEQ ID NOS. 6 or 8, or a residue at a position corresponding to position 668 of SEQ ID NOS. 2 or 4.

3. A method according to claim 2, wherein the presence of an Arginine residue at said position is indicative of an individual at risk of suffering from or developing a heart condition post myocardial infarction (MI).

4. A method according to claim 2, wherein the presence of a Glutamine residue at said position is indicative of a protective effect.

5. A method according to claim 1, wherein the identity of a SNP (A/G) is detected at a position corresponding to position 443 of SEQ ID NOS 5 or 7, or at a position corresponding to position 7265 of SEQ ID NOS. 1 or 3.

6. A method according to claim 1, wherein the sequence detected is a polynucleotide selected from the group consisting of SEQ ID NOS 1, 3, 5, and/or 7.

7. A method according to claim 1, wherein the sequence detected is an amino acid sequence selected from the group consisting of SEQ ID NOS 2, 4, 6, and/or 8.

8. A method according to claim 4, wherein the presence of a Glutamine amino acid residue at said position is indicative of a high Ejection Fraction (EF), low early phase MMP-9 activity, post Myocardial Infarction, and a reduced chance of developing heart failure.

9. A method according to claim 8, wherein the low early phase MMP-9 is measured up to 24 hours after the individual has suffered a Myocardial Infarction.

10. A screening method for infarcted patients, comprising determining the presence or absence of the amino acid change in the sequence of the hemopexin domain of MMP-9, as defined in claim 1.

11. A screening method for an uninfarcted population, comprising determining the presence or absence of the amino acid change, as defined in claim 1, in the sequence of the hemopexin domain of MMP-9.

12. A method of establishing a diagnosis and/or a prognosis in patients with myocardial infarction, by correlating an amino acid change in the sequence of the hemopexin domain of MMP-9, as defined in claim 1, with lower levels of MMP-9 activity, which indicates a better clinical outcome after myocardial infarction.

13. A method of treatment of inhibiting ventricular remodelling and the treatment and/or prophylaxis of heart failure by correlating an amino acid change in the hemopexin domain of matrix-metalloproteinase-9 (MMP-9), as defined in claim 1, with lower MMP-9 levels, which indicates a better clinical outcome after myocardial infarction.

14. A method of predicting the occurrence of ventricular remodelling in patients following myocardial infarction, by correlating an amino acid change in the hemopexin domain of matrix-metalloproteinase-9 (MMP-9), as defined in claim 1, with lower MMP-9 levels, which indicates a better clinical outcome after myocardial infarction.

15. A method of identifying agonists or antagonists/inhibitors for MMP-9, or molecules capable of blocking MMP-9 activity, or reinforcing its interaction with TIMP-1 or reducing the detectable activity or expression of MMP-9, comprising designing a molecule or ligand that will interact with the changed hemopexin domain of matrix-metalloproteinase-9 (MMP-9), as defined in claim 1.

16. A method for assaying for the presence of a first polynucleotide having a SNP associated with susceptibility to a heart condition in a sample, comprising;
contacting said sample with a second polynucleotide, wherein said second polynucleotide comprises a nucleotide sequence selected from the group consisting SEQ. ID. NOS.: 1,3, 5, 7, 9 and/or 10 and the complements of said sequences, wherein said second polynucleotide hybridizes to said first polynucleotide under stringent conditions.

17. A method of assaying for the presence of a polypeptide encoded by an isolated polynucleotide having a SNP in the hemopexin domain of MMP-9 associated with susceptibility to a heart condition in a sample, said method comprising contacting the sample with an antibody which specifically binds to said encoded polypeptide.

18. A method of identifying an agent that alters expression of a polynucleotide containing a SNP associated with susceptibility to a heart condition comprising:
(a) contacting a polynucleotide with an agent to be tested under conditions for expression, wherein the polynucleotide comprises (1) a SNP in the hemopexin domain of MMP-9 associated with susceptibility to a heart condition and (2) a promoter region operably linked to a reporter gene;
(b) assessing the level of expression of the reporter gene in the presence of the agent;
(c) assessing the level of expression of the reporter gene in the absence of the agent; and
(d) comparing the level of expression in step (b) with the level of expression in step (c) for differences which indicate that expression was altered by the agent.

19. A method of diagnosing susceptibility to a heart condition in an individual, comprising:
a) obtaining a polynucleotide sample from said individual; and
b) analyzing the polynucleotide sample for the presence or absence of a haplotype, wherein the presence of the haplotype corresponds to a susceptibility to said heart condition; wherein the haplotype comprises an allele resulting from a SNP in the hemopexin domain of MMP-9 associated with susceptibility to a heart condition.

20. A method according to claim 1, wherein the heart condition is at least one selected from the group consisting of:
myocardial infarction, acute coronary syndrome, ischemic cardiomyopathy, non-ischemic cardiomyopathy and congestive heart failure.

21. A method according to claim 20, wherein the heart condition is congestive heart failure.

22. A vector comprising an isolated polynucleotide containing a SNP in the hemopexin domain of MMP-9 associated with susceptibility to a heart condition, wherein said isolated polynucleotide is operably linked to a regulatory sequence, preferably a suitable promoter.

23. A host cell comprising the vector according to claim 22.

24. A transgenic animal comprising a polynucleotide having a SNP in the hemopexin domain of MMP-9 associated with susceptibility to a heart condition.

25. A kit for assaying a sample for the presence of a first polynucleotide comprising a SNP in the hemopexin domain of MMP-9 associated with susceptibility to a heart condition, comprising in separate containers:
a) one or more labelled second polynucleotides comprising a sequence selected from the group consisting of sequences identified by SEQ. ID. NOS.: 1,3, 5, 7, 9 and/or 10, and the complements thereof; and
b) reagents for detection of said label.
